(19) United States
(12) Patent Application Publication
(10) Pub. No.: US 2014/0005719 A1
(43) Pub. Date: Jan. 2, 2014

(54) SUTURE NEEDLE

(71) Applicants: Tsuyoshi Uetake, Bunkyo-ku (JP); Kenjiro Hasegawa, Okayama-shi (JP)

(72) Inventors: Tsuyoshi Uetake, Bunkyo-ku (JP); Kenjiro Hasegawa, Okayama-shi (JP)

(73) Assignee: KEISEI MEDICAL INDUSTRIAL CO., LTD., Bunkyo-ku, Tokyo (JP)

(21) Appl. No.: 13/976,864

(22) PCT Filed: Dec. 26, 2011
(86) PCT No.: PCT/JP2011/080013

§ 371 Date: Sep. 18, 2013

(30) Foreign Application Priority Data
Dec. 28, 2010 (JP) 2010-291554
Dec. 20, 2011 (JP) 2011-277959

(51) Int. Cl. A61B 17/06

(52) U.S. Cl. CPC A61B 17/06066
USPC 606/223

(57) ABSTRACT

An object of the present invention is to provide a revolutionary suture needle of exceptionally high marketability. The suture needle curves in the lengthwise direction of the needle body. A curved inner surface portion extending from a needle distal end (3) to a predetermined position P1 towards the basal end of a distal end portion (2) of circular cross section is furnished with frictional resistance reducing means for reducing the frictional resistance to less than that of a curved outer surface portion from the needle distal end (3) to the predetermined position P1 towards the basal end. The frictional resistance reducing means is a chamfered portion (4) chamfered at a surface passing through the needle distal end (3). The needle distal end (3) is furnished at a position coincident with the center axis L of the needle body (1).

### TECHNICAL FIELD

The present invention relates to a suture needle for use in surgical procedures for example, having a curved region in the lengthwise direction of the needle body.

### BACKGROUND ART

The applicant previously proposed a suture needle disclosed, for example, in Japanese Laid-Open Patent Application 2000-139931 (herein termed “prior art example”), in which the cross section of the needle body is given a polygonal shape, as a suture needle for use in surgical procedures, having a curved region in the lengthwise direction of the needle body.

According to this prior art example, the needle body is given a polygonal shape in which each edge is a blade, thereby improving the ease of entry into body tissue. In more detail, during entry into body tissue, due to the presence of the plurality of edges (blades) entry takes place in a manner that cuts into the body tissue, thereby affording extremely good penetration, as well as minimizing the area of contact with the region of entry, thereby reducing the resistance. The suturing procedure can therefore be carried out with minimal impact on body tissue.

### PRIOR ART DOCUMENT

### Patent Document

(Patent Document 1) Japanese Laid-Open Patent Application 2000-139931

### DISCLOSURE OF THE INVENTION cl Problem the Invention is Intended to Solve

The walls of bodily vasculature, such as blood vessels or lymph vessels, have a suitable degree of rigidity. Consequently, the prior art example is found to have the problem that, in cases of suturing the wall of such a vessel, there are cases in which the region being sutured becomes damaged.

As a result of additional research and testing of suture needles having a curved region in the lengthwise direction of the needle body, the inventors developed a revolutionary suture needle exhibiting working effects that are lacking in the prior art.

### Means for Solving the Problem

The gist of the present invention will be described with reference to the accompanying drawings.

The present invention in a first aspect relates to a suture needle having a curved region in a lengthwise direction of a needle body 1, wherein the suture needle is characterized in that a curved lengthwise inner surface portion from a needle distal end 3 to a predetermined position P1 towards a basal end of a distal end portion 2 of circular cross section is furnished with frictional resistance reducing means for reducing the frictional resistance to less than that of a curved lengthwise outer surface portion.

The present invention in another aspect relates to the suture needle according to the first aspect, wherein the suture needle is characterized in that the frictional resistance reducing means is a chamfered portion 4 furnished from the needle distal end 3 to the predetermined position P1 towards the basal end of to the curved lengthwise inner surface portion, and chamfered at the surface thereof passing through the needle distal end 3.

The present invention in another aspect relates to the suture needle according to the second aspect, wherein the suture needle is characterized in that the chamfered portion 4 is established on a concavely curved surface in the lengthwise direction.

The present invention in another aspect relates to the suture needle according to the second aspect, wherein the suture needle is characterized in that the chamfered portion 4 is established on a concavely curved surface in the widthwise direction.

The present invention in another aspect relates to the suture needle according to the third aspect, wherein the suture needle is characterized in that the chamfered portion 4 is established on a concavely curved surface in the widthwise direction.

The present invention in another aspect relates to the suture needle according to the second aspect, wherein the suture needle is characterized in that side edge blades 5 continuous with the needle distal end 3 are furnished at the left and right side edges of the chamfered portion 4.

The present invention in another aspect relates to the suture needle according to the third aspect, wherein the suture needle is characterized in that side edge blades 5 continuous with the needle distal end 3 are furnished at the left and right side edges of the chamfered portion 4.

The present invention in another aspect relates to the suture needle according to the fourth aspect, wherein the suture needle is characterized in that side edge blades 5 continuous with the needle distal end 3 are furnished at the left and right side edges of the chamfered portion 4.

The present invention in another aspect relates to the suture needle according to the fifth aspect, wherein the suture needle is characterized in that side edge blades 5 continuous with the needle distal end 3 are furnished at the left and right side edges of the chamfered portion 4.

The present invention in another aspect relates to the suture needle according to any of the second to ninth aspects, wherein the suture needle is characterized in that the chamfered portion 4 is formed by cutting, press working, or die forming.

The present invention in another aspect relates to the suture needle according to the first aspect, wherein the suture needle is characterized in that the frictional resistance reducing means is a smooth surface of lower frictional resistance than the curved lengthwise outer surface portion, furnished from the needle distal end 3 to the predetermined position P1 towards the basal end of to the curved lengthwise inner surface portion.

The present invention in another aspect relates to the suture needle according to any of the first to ninth aspects, wherein the suture needle is characterized in that the needle distal end 3 is furnished at a position coincident with the center axis L of the needle body 1.

The present invention in another aspect relates to the suture needle according to the tenth aspect, wherein the suture needle is characterized in that the needle distal end 3 is furnished at a position coincident with the center axis L of the needle body 1.

The present invention in another aspect relates to the suture needle according to the eleventh aspect, wherein the suture needle is characterized in that the needle distal end 3 is furnished at a position coincident with the center axis L of the needle body 1.

### Effect of the Invention

The present invention is configured in the above manner, and therefore as compared with the prior art example mentioned previously, affords a revolutionary suture needle endowed with suitable ease of penetration, and with excellent operability during suturing, making it extremely suited to suturing the walls of bodily vasculature, such as blood vessels or lymph vessels for example.

### BRIEF DESCRIPTION OF THE DRAWINGS

FIG. 1 is a perspective view showing a first example;

FIG. 2 is a side view showing the first example;

FIG. 3 is a perspective view showing a major part according to the first example;

FIG. 4 is a side view showing a major part according to the first example;

FIG. 5 is an V-V cross sectional view of FIG. 3;

FIG. 6 is a descriptive diagram of an incision in body tissue where the suture needle according to the first example has entered;

FIG. 7 is a perspective view showing a major part according a suture needle (round needle) proposed in the prior art;

FIG. 8 is a VIII-VIII cross sectional view of FIG. 7;

FIG. 9 is a descriptive diagram of an incision made in body tissue by entry of a suture needle proposed in the prior art;

FIG. 10 is a perspective view showing a major part according a suture needle (angled needle) proposed in the prior art;

FIG. 11 is a XI-XI cross sectional view of FIG. 10;

FIG. 12 is a descriptive diagram of operation during entry of a suture needle proposed in the prior art into body tissue;

FIG. 13 is a descriptive diagram of an incision made in body tissue by entry of a suture needle proposed in the prior art;

FIG. 14 is a perspective view showing a major part according to a second example;

FIG. 15 is a side view showing a major part according to the second example;

FIG. 16 is a plan view showing a major part according to the second example;

FIG. 17 is a XVII-XVII cross sectional view of FIG. 14;

FIG. 18 is a cross sectional view of another example;

FIG. 19 is a perspective view showing a major part according to a third example;

FIG. 20 is a side view showing a major part according to the third example;

FIG. 21 is a plan view showing a major part according to the third example; and

FIG. 22 is an XXII-XXII cross sectional view of FIG. 19.

### BEST MODE FOR CARRYING OUT THE INVENTION

Preferred embodiments of the present invention are briefly described below with reference to the diagrams while indicating the effects of the present invention.

During entry into body tissue, due to the presence of the frictional resistance reducing means on the curved lengthwise inner surface portion from the needle distal end 3 to the predetermined position P1 towards the basal end, the contact resistance with respect to the body tissue is minimal, whereby the needle enters successfully while giving rise to an appropriate level of resistance so as to not create excessive resistance, and furthermore enters successfully on a trajectory aligned with the curved lengthwise region of the needle body 1.

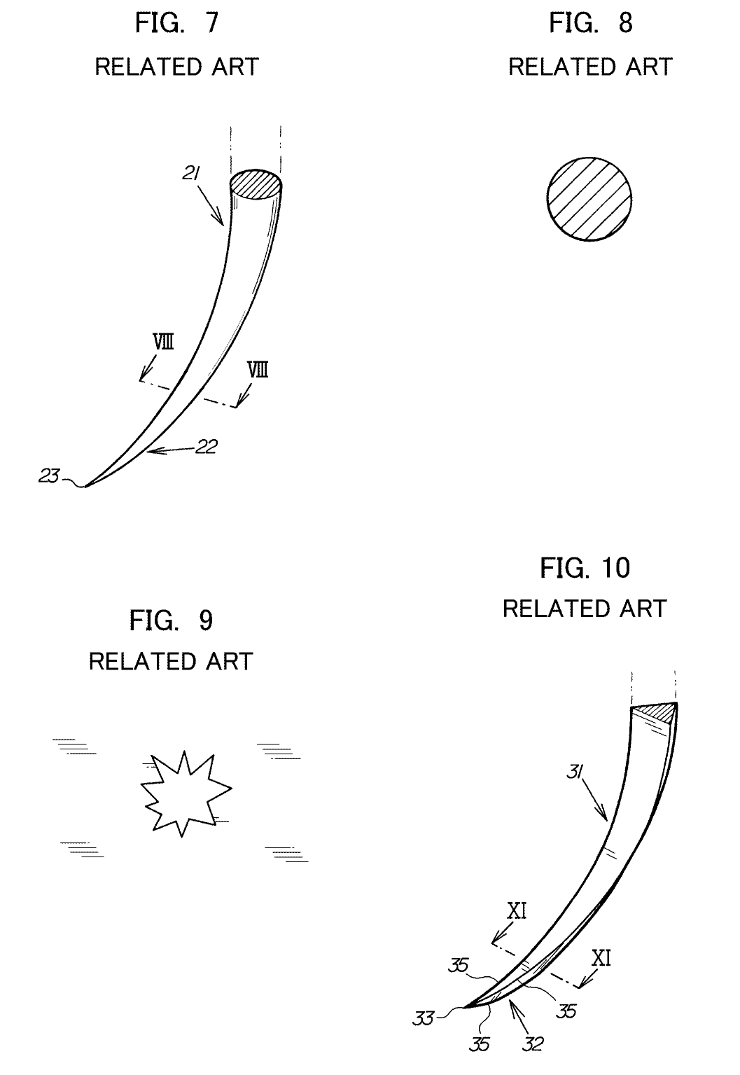

Specifically, because the suture needle according to the present invention is furnished, on the curved lengthwise inner surface portion from the needle distal end 3 to the predetermined position P1 of the distal end portion 2 of circular cross section, with the frictional resistance reducing means for reducing the frictional resistance to less than that of the curved lengthwise outer surface portion, the needle sticks through more easily as compared, for example, with the case in which the distal end portion 22 of the needle body 21 has a round conical structure (structure of circular cross section) from the needle distal end 23 as shown in FIGS. 7 and 8, and is moreover not furnished with frictional resistance reducing means such as the aforedescribed.

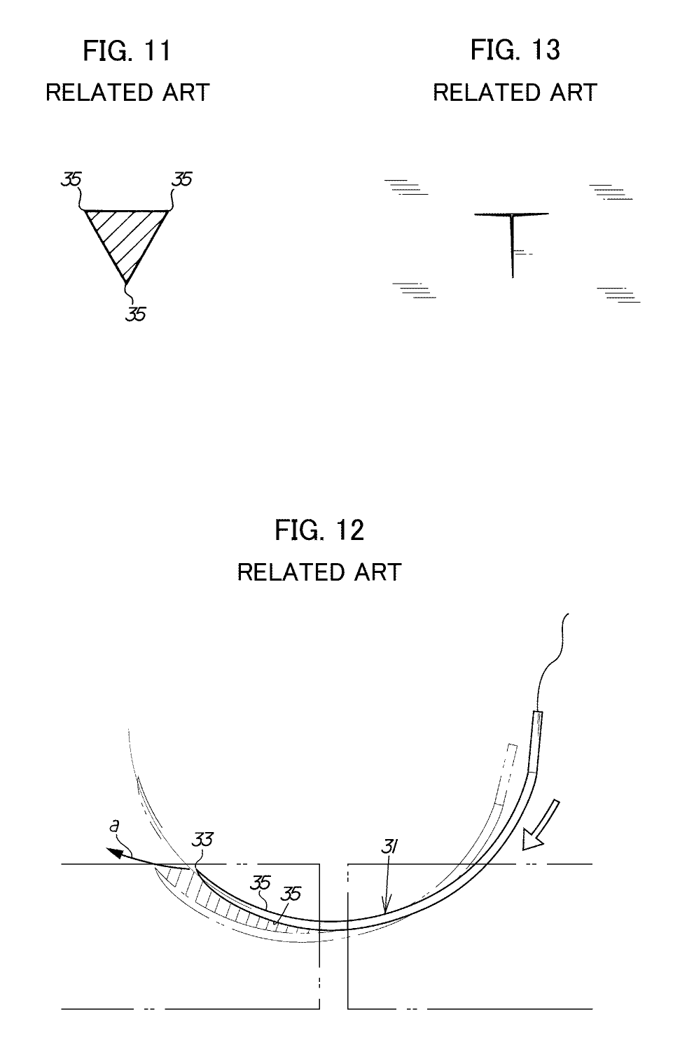

Moreover, as compared with the case of a structure in which the distal end portion 32 of the needle body 31 is furnished with a plurality (three) blade edges 35 continuous with the needle distal end 33 (structure of triangular cross section), as shown in FIGS. 10 and 11, the needle sticks through in an appropriate manner, so as to not stick through excessively.

In specific terms, in the case in which the needle body 1 having a curved lengthwise region enters into body tissue, pushing force towards the forward side in the direction of entry is applied to the needle body 1, until the time that the needle distal end 3 enters the body tissue and the needle body 1 penetrates through the body tissue. Consequently, in the case of a structure in which the curved lengthwise outer surface portion has blade edges 35 as shown in FIGS. 10 and 11 for example, because the blade edges 35 of the curved lengthwise outer surface portion cut into body tissue towards the forward side in the direction of entry, they tend to deviate in the outward direction of the curve (the direction shown by arrow a in FIG. 12) from a trajectory aligned with the curved lengthwise region of the needle body 1, and therefore, as compared with a structure in which the distal end portion 22 of the needle body 21 has a circular cross section, there are cases in which the needle does not advance along the intended trajectory, causing damage to body tissue.

In this regard, according to the present invention, because the curved lengthwise inner surface portion has the frictional resistance reducing means, a frictional resistance differential arises between the curved lengthwise inner surface portion and the curved lengthwise outer surface portion during entry into body tissue. Specifically, the frictional resistance of the curved lengthwise inner surface portion where the frictional resistance reducing means is present is lower as compared with that of the curved lengthwise outer surface portion, and therefore the needle body 1 does not readily advance towards the outward direction of the curved lengthwise region (while advancing easily towards the inward direction of the curved lengthwise region of low frictional resistance), and moreover, due to the lack of blade edges of the curved lengthwise outer surface portion, does not cut into body tissue towards the forward side in the direction of entry. Consequently, there is no deviation from a trajectory aligned with the curved lengthwise region of the needle body 1, thus minimizing damage to body tissue as much as possible. Furthermore, the characteristic that the needle body 1 does not readily deviate from its intended trajectory provides good operability during suturing.

That is, the present invention affords reduced contact resistance with respect to body tissue, to achieve successful entry, while preserving the advantages afforded by a structure in which the distal end portion of the needle body has a circular cross section so as to not readily damage body tissue.

A structure in which the distal end portion 22 of the needle body 21 has a circular cross section creates an entry hole of ragged shape in body tissue as illustrated in FIG. 9, whereas a structure in which the distal end portion 32 of the needle body 31 has a triangular cross section creates a “T” shaped entry hole in body tissue as illustrated in FIG. 13, leading to the problem of significant damage to body tissue.

In contrast to this, due to the lack of blade edges in the curved lengthwise outer surface portion, the suture needle according to the present invention creates a straight-line entry hole in body tissue, as illustrated in FIG. 6, thereby reducing damage to body tissue as much as possible when the suture needle according to the present invention is used.

An appropriate manner of cutting such that body tissues are not overloaded is obtained thereby, and a successful suturing operation can be performed. It has been verified, for example, that in actual practice, walls of bodily vasculature can be successfully sutured by the suture needle according to the present invention.

### Example 1

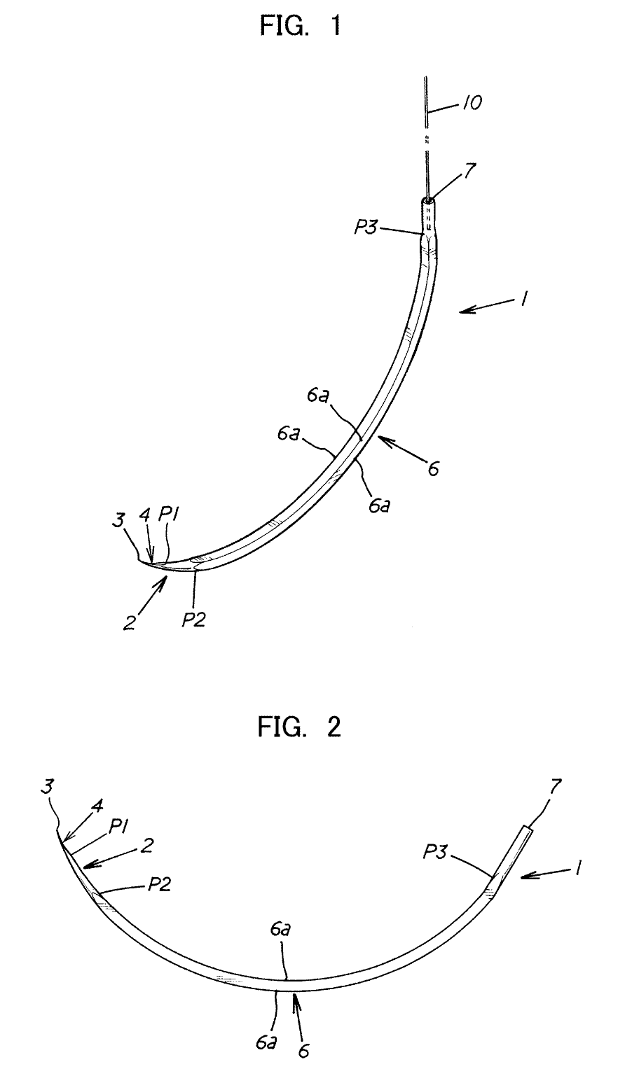

A specific first example of the present invention is described below on the basis of FIGS. 1 to 5.

The present example is a suture needle for use in surgical procedure.

In specific terms, as shown in FIGS. 1 and 2, the needle body 1 is formed by curving a rod member made of an appropriate metal into a bow shape, and has a curved lengthwise region lying between the needle distal end 3 of a distal end portion 2 of circular cross section (conical shape) and a predetermined position P3 towards the basal end.

The basal end of the needle body 1 is furnished with a linear connector portion 7 (tubular portion) having a circular cross section for threading and connecting a suture yarn 10.

The needle distal end 3 is furnished at a position coincident with the center axis L of the needle body 1 (see FIG. 4).

Consequently, due to this constitution, entry into body tissue takes place through cutting in a state in which uniform pressure is applied to left and right side edge blades 5 (discussed below) centered on the needle distal end 3, thereby reducing the load on body tissue as much as possible.

On the curved lengthwise inner surface portion from the needle distal end 3 of the distal end portion 2 to a predetermined position P1 towards the basal end, the needle body 1 is furnished with frictional resistance reducing means for reducing the frictional resistance to less than that of the curved lengthwise outer surface portion.

As shown in FIGS. 1 to 3, this frictional resistance reducing means is a chamfered portion 4 furnished from the needle distal end to a predetermined position P1 towards the basal end on the curved lengthwise inner surface portion, and chamfered at the surface thereof that passes through the needle distal end 3.

This chamfered portion 4 is a sloped surface (or a concavely-curved lengthwise surface) passing through the needle distal end 3, and has been furnished by cutting (machining) the needle body 1. Optionally, this chamfered portion 4 is furnished by press working or die forming.

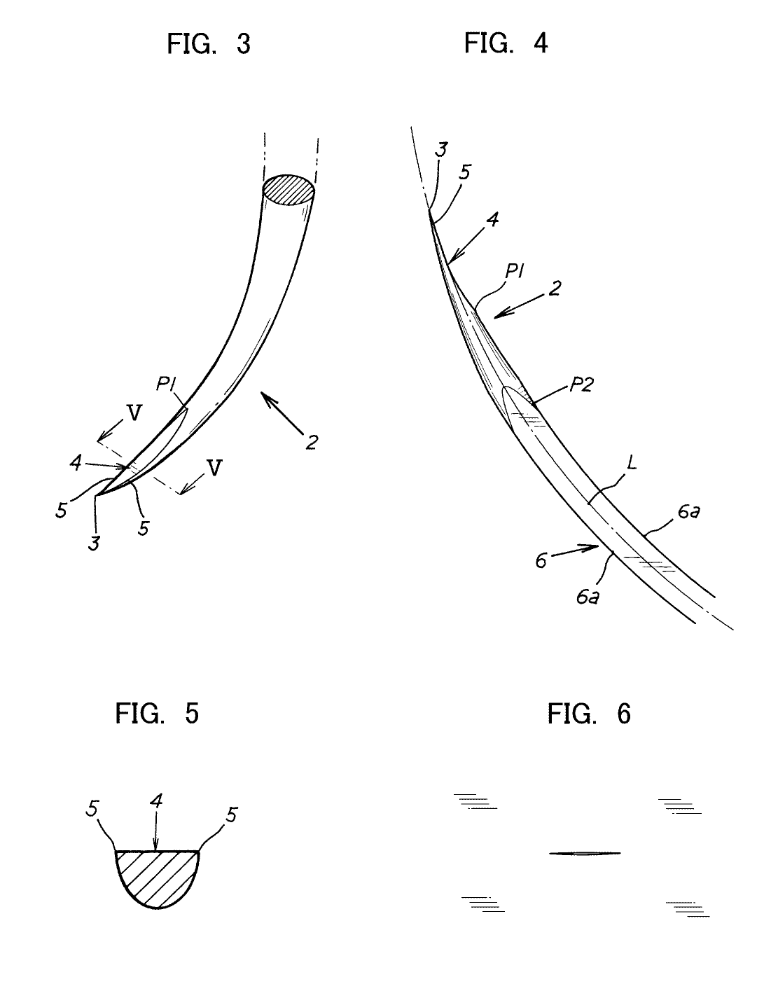

The chamfered portion 4 is shaped like an ellipse that is sharp-pointed at one end in plan view as illustrated in FIG. 3. Side edge blades 5 continuous with the needle distal end 3 are furnished at the left and right side edges of this chamfered portion 4. That is, the distal end portion 2 of the needle body 1 is furnished with the pair of side edge blades 5.

Consequently, the cross section of the region furnished with this chamfered portion 4 is shaped like a semicircle having a flat surface in the top part (the curved lengthwise inner surface portion), and curving at the bottom part (the curved lengthwise outer surface portion), as illustrated in FIG. 5. Optionally, the top surface of the chamfered portion 4 (the surface between the side edge blades 5) may be formed as a concavely-curved widthwise surface.

In the present example, this chamfered portion 4 is furnished exclusively at the distal end of the distal end portion 2 of the needle body 1 as described previously; however, the chamfered portion 4 may be extended so as to continue from the distal end to the basal end of the needle body 1. However, considerations pertaining to strength and to workability make it preferable for the chamfered portion 4 to be shorter in length; moreover, considerations pertaining to ease of penetration suggest that it is more effective for the chamfered portion 4 to be shorter, while maintaining a gentle slope.

Specifically, to the extent that the chamfered portion 4 is extended in length, the sideways width of the chamfered portion 4 expands, blunting the sharpness of the needle tip, and therefore a shorter chamfered portion 4 means that it will be easier to preserve the sharpness of the needle tip. In the present example, by making the chamfered portion 4 shorter, the region to the basal end side from the region furnished with the chamfered portion 4 is given a circular cross section. During entry into body tissue, the region extending from the needle distal end 3 to the basal end of the chamfered portion 4 having the side edge blades 5 (the predetermined position P1 towards the basal end) enters sharply; next, due to the presence of the region of circular cross section extending from the predetermined position P1 towards the basal end to a predetermined position P2 towards the basal end, an appropriate manner of entry so as to not sticking through excessively is achieved; and next, due to the presence of side edge blades 6a of a polygonal cross section portion 6, discussed later, that extends from the predetermined position P2 towards the basal end to a predetermined position P3 towards the basal end, the needle enters sharply, so that the needle body 1 penetrates successfully through the wall of the body tissue, with attendant changes in the manner of entry in each of these regions of the needle.

In the present example, the predetermined position P1 towards the basal end is a position equivalent to 7% of the total length of the needle body 1 from the needle distal end 3, the predetermined position P2 towards the basal end is a position equivalent to 16%, and the predetermined position P3 towards the basal end is a position equivalent to 82%.

Testing has shown that it is preferable for the predetermined position P1 towards the basal end to be about 5 to 10% of the total length of the needle body 1, and for the predetermined position P2 towards the basal end to be about 10 to 30%.

It has been verified that the shape of the region extending between the predetermined position P2 towards the basal end and the predetermined position P3 towards the basal end does not have much of an effect on performance.

Optionally, the frictional resistance reducing means may be a smooth surface of lower frictional resistance than the curved lengthwise outer surface portion, furnished to the curved lengthwise inner surface portion in the region extending from the needle distal end 3 to the predetermined position P1 towards the basal end.

This smooth surface may be created by supplying the curved inner lengthwise surface portion with a smooth surface material of fluid form or sheet form, or by a polishing process to polish the curved inner lengthwise surface portion; these methods can serve to reduce the frictional resistance of the distal end portion 2 of the needle body 1 without lowering the strength.

Optionally, the frictional resistance reducing means may be a recessed portion furnished to the curved lengthwise inner surface portion in the region extending from the needle distal end 3 to the predetermined position P1 towards the basal end, having a bottom surface that passes through the needle distal end 3, and having length in the lengthwise direction of the needle body 1.

This recessed portion furnished through cutting (machining), press working, or die forming of the needle body 1.

As yet another frictional resistance reducing means, for example, the curved inner lengthwise surface portion and the curved outer lengthwise surface portion constituting the distal end portion 2 of the needle body 1 may be different materials (for example, different metals, or a metal and an resin), thereby reducing the frictional resistance of the curved inner lengthwise surface portion to below the frictional resistance of the curved outer lengthwise surface portion.

In the present example, a polygonal cross section portion 6 is formed in a curved region that is a region to the basal end side from the aforementioned chamfered portion 4.

In the present example, the polygonal cross section portion 6 is constituted by forming a square cross section portion extending from the predetermined position P2 towards the basal end to the predetermined position P3 towards the basal end of the needle body 1. Consequently, the curved inner and outer surface portions extending from the predetermined position P2 towards the basal end to the predetermined position P3 towards the basal end of the needle body 1 are furnished with four side edge blades 6a having length in the length direction of the needle body 1.

The surfaces between the side edge blades 6a in this polygonal cross section portion (square cross section portion) have been formed into concavely-curved surfaces.

Optionally, a polygonal cross section portion of some other shape (a triangular cross section portion, a pentagon cross section portion, a hexagon cross section portion, or the like) may be disposed parallel to the lengthwise direction of the needle body 1.

In the present example, by virtue of the constitution discussed above, during entry of the needle into body tissue, the curved inner lengthwise surface portion extending from the needle distal end 3 to the predetermined position P1 towards the basal end has minimal contact resistance with respect to the body tissue due to the presence of the frictional resistance reducing means, whereby the needle enters successfully while giving rise to an appropriate level of resistance so as to not create excessive resistance, and furthermore enters successfully on a trajectory aligned with the curved region of the needle body 1.

Therefore, as compared with the prior art example mentioned previously, the needle according to the present example does is endowed with suitable ease of penetration so as to not stick through excessively, making it extremely suited to suturing the walls of bodily vasculature, such as blood vessels or lymph vessels for example.

Moreover, in the present example, because the curved inner lengthwise surface portion extending from the needle distal end 3 to the predetermined position P1 towards the basal end has been furnished with the chamfered portion 4 as the frictional resistance reducing means, the needle body 1 enters successfully with minimal frictional resistance, and moreover, a differential in contact resistance arises between the inner side and the outer side of the curved needle body 1, and at the inner side of the needle body 1, where the contact resistance is minimal, the entering action (the entering action in the in-curve direction) takes place in an active manner, and therefore penetration of the needle body 1 through the wall of the body tissue takes place in an exceptionally smooth manner.

Moreover, in the present example, because the chamfered portion 4 is a sloped surface, and has been formed by cutting, press working, or die forming, a structure whereby the outstanding working effects discussed previously are realized is obtained in a simple and reliable manner.

Moreover, in the present example, because the needle distal end 3 has been furnished at a position coincident with the center axis L of the needle body 1, entry into body tissue takes place through cutting in a state in which uniform pressure is applied to the left and right side edge blades 5 centered on the needle tip, thereby reducing the load on body tissue as much as possible.

### Example 2

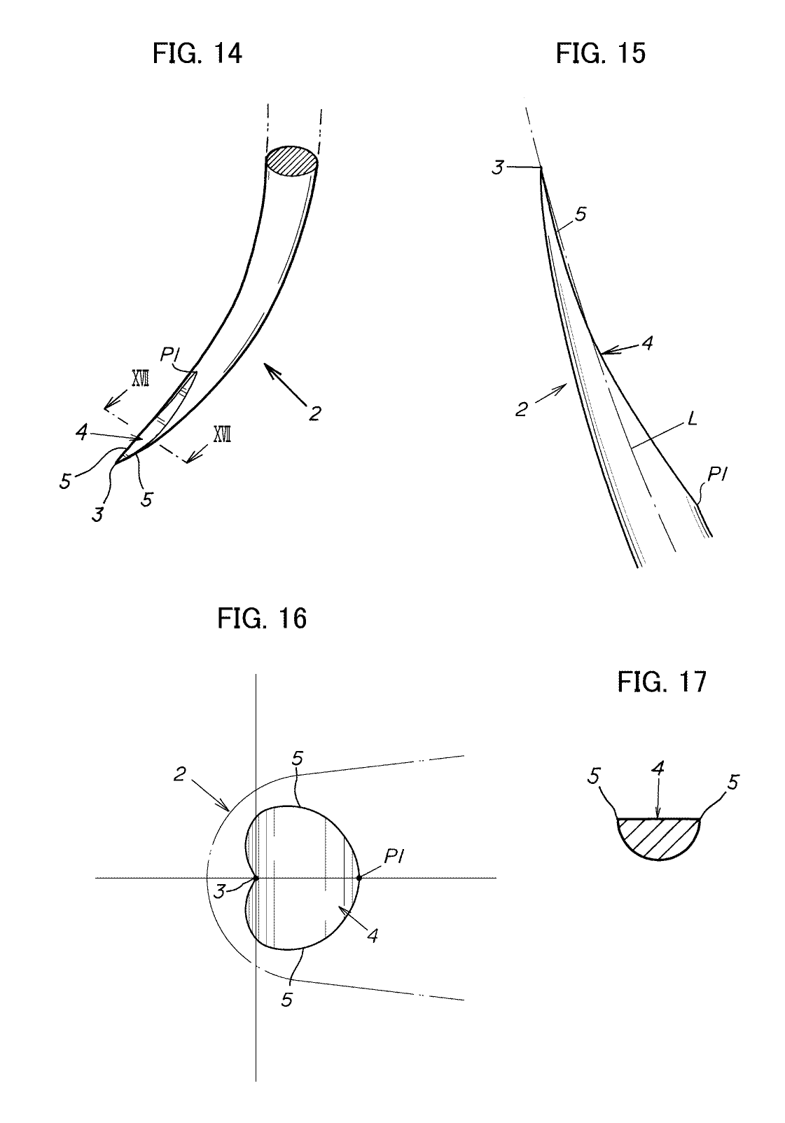

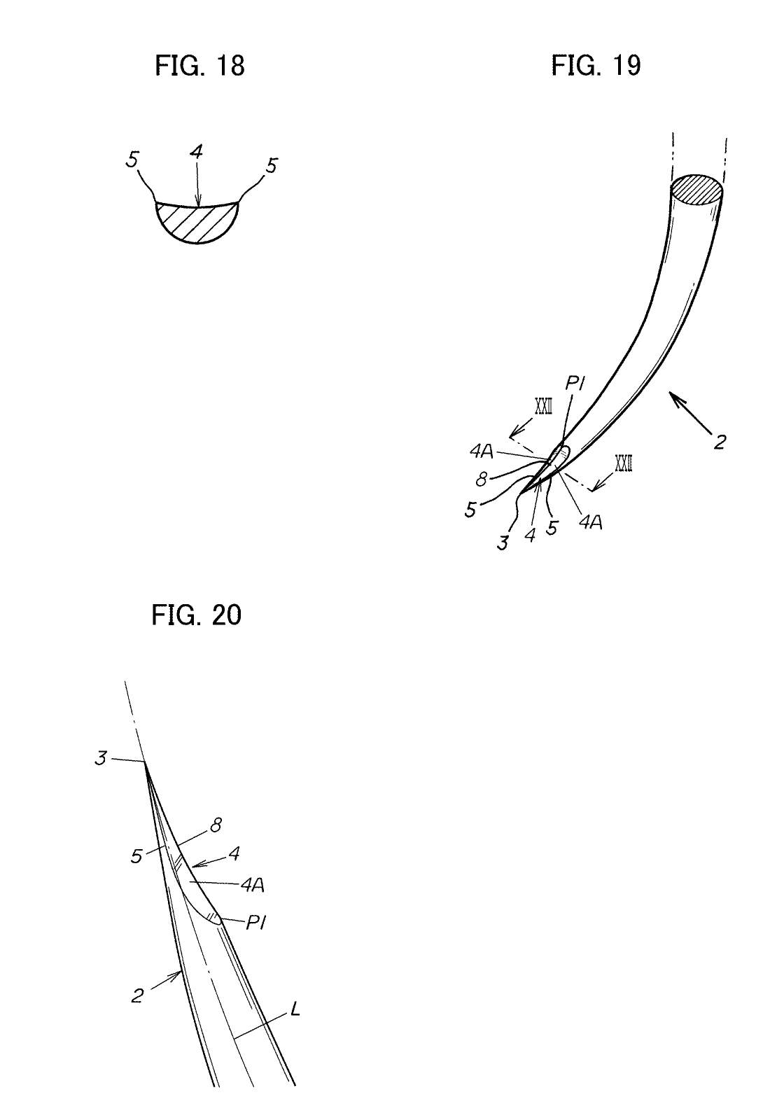

A specific second example of the present invention is described below on the basis of FIGS. 14 to 18.

The present example is a case in which a portion of the chamfered portion 4 (a region to the distal end side) has been formed to greater depth than the center axis L of the needle body 1, as illustrated in FIGS. 14 and 15. FIG. 17 depicts a case in which the upper surface of the chamfered portion 4 is formed to a concavely-curved widthwise surface.

In other respects, the design is comparable to Example 1.

### Example 3

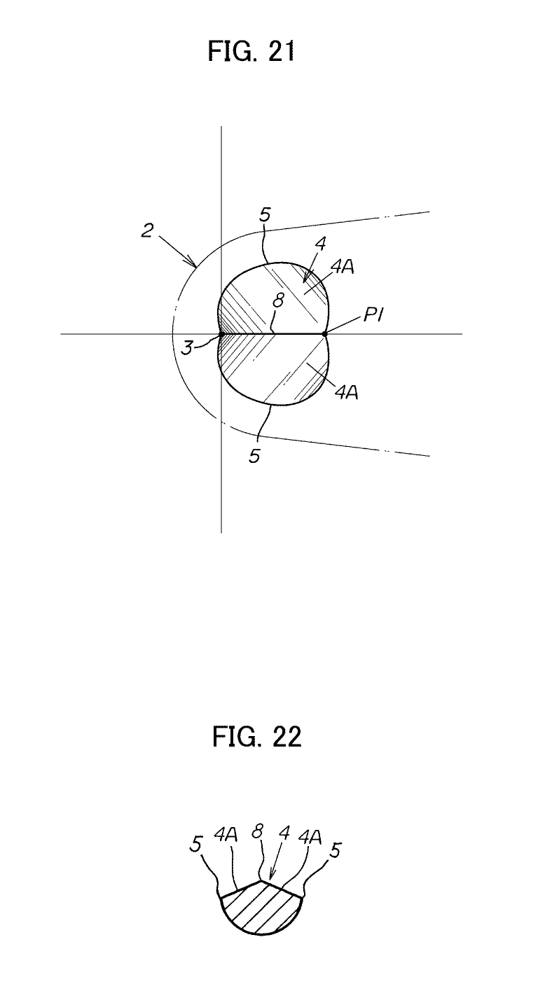

A specific third example of the present invention is described below on the basis of FIGS. 19 to 22.

According to the present example, the chamfered portion 4 is constituted by left and right descending surfaces 4A, 4A that either incline downwardly or concavely curve towards the left and right directions from a boundary (ridge portion) at the center between the left and right side edge blades 5. A center blade 8 is furnished in the lengthwise direction of the needle body 1, at the connecting region between these descending surfaces 4A, 4A.

In other respects, the design is comparable to Example 1 and 2.

The present invention is not limited to the present example, and specific constitutions of the constituent elements thereof may be redesigned, as appropriate.

1. A suture needle that curves in a lengthwise direction of a needle body, wherein the suture needle is characterized in that a curved inner surface portion from a needle distal end to a predetermined position towards a basal end in a distal end portion of circular cross section is furnished with frictional resistance reducing means for reducing the frictional resistance to less than that of a curved outer surface portion from the needle distal end to the predetermined position towards the basal end; the frictional resistance reducing means is a chamfered portion chamfered at a surface passing through the needle distal end; and the needle distal end is furnished at a position coincident with a center axis of the needle body.

2. The suture needle according to claim 1, wherein the suture needle is characterized in that the predetermined position towards the basal end is a position that is 5 to 10% of the total length of the needle body from the needle distal end.

3. The suture needle according to claim 1 or 2, wherein the suture needle is characterized in that the chamfered portion is established on a concavely curved surface in the lengthwise direction.

4. The suture needle according to claim 1 or 2, wherein the suture needle is characterized in that the chamfered portion is established on a concavely curved surface in the widthwise direction.

5. The suture needle according to claim 3, wherein the suture needle is characterized in that the chamfered portion is established on a concavely curved surface in the widthwise direction.

6. The suture needle according to claim 1 or 2, wherein the suture needle is characterized in that side edge blades continuous with the needle distal end are furnished at the left and right side edges of the chamfered portion.

7. The suture needle according to claim 3, wherein the suture needle is characterized in that side edge blades continuous with the needle distal end are furnished at the left and right side edges of the chamfered portion.

8. The suture needle according to claim 4, wherein the suture needle is characterized in that side edge blades continuous with the needle distal end are furnished at the left and right side edges of the chamfered portion.

9. The suture needle according to claim 5, wherein the suture needle is characterized in that side edge blades continuous with the needle distal end are furnished at the left and right side edges of the chamfered portion.

10. The suture needle according to claim 1 or 2, wherein the suture needle is characterized in that the chamfered portion is formed by cutting, press working, or die forming.

11-14. (canceled)
